(19) United States
(12) Patent Application Publication
(10) Pub. No.: US 2019/0214119 A1
(43) Pub. Date: Jul. 11, 2019

(54) SYSTEM AND METHOD FOR PERSONALIZING AND OPTIMIZING MEDICATION REGIME

(71) Applicant: International Business Machines Corporation, Armonk, NY (US)

(72) Inventors: Charles Muchiri Wachira, Nairobi (KE); Osebe Mogaka Samuel, Nairobi (KE); John Mbari Wamburu, Nairobi (KE); Aisha Walcott, Nairobi (KE); Sekou Lionel Remy, Nairobi (KE); Katherine Anne Lyon Tryon, Nairobi (KE)

(21) Appl. No.: 15/863,044

(22) Filed: Jan. 5, 2018

(51) Int. Cl. G16H 20/10; G16H 40/63

(52) U.S. Cl. CPC G16H 20/10; G06Q 10/109; G16H 40/63

(57) ABSTRACT

Methods are provided for personalizing and optimizing a medication regime for a patient. For example a method involves generating a medication regime for one or more prescribed medications associated with a patient as input via a user interface; personalizing the generated medication regime for the patient via the user interface; generating a regime planner from the personalized medication regime for the patient via the user interface; and forwarding a reminder to the patient to take the prescribed one or more medications from the generated regime planner via the user interface, wherein the steps of the method are performed in accordance with a processor and a memory.

### BACKGROUND

This disclosure generally relates to a system and method for personalizing and optimizing a medication regime for a user.

Poor patient adherence to taking and complying with treatment regimens is a major concern within the health care industry. After visiting their health care provider and receiving at least one prescription for medication such as a pharmaceutical substance, many patients thereafter fail to maintain a level of compliance with their prescribed treatment regimen and adherence to diligently taking their mediation as prescribed and/or continuing their medication regimen by timely seeking refills on their medication. For example, a failure to maintain a level of compliance includes lack of personalization of medication regimes to a patient's preferences and contexts especially for polypharmacy patients.

From the pharmacy, a patient at a minimum is able to obtain at least one prescription for medication, dosage information, frequency and the directions of use (e.g., take on an empty stomach, take after a meal, etc.). However, when it comes to taking the medication, the patient either changes their activities of daily living to suite taking the drugs, or changes the time to take the medication which might not agree with the specified half-life of the medication, or they forget to take medication or stop adhering altogether. This practice results in increased costs to all parties involved in the health care industry, including, but not limited to the patient, the health care provider, the pharmacies, the pharmaceutical companies, and the health insurance companies.

### SUMMARY

Embodiments described herein provide a system and method for personalizing and optimizing medication regime for a patient.

For example, one exemplary embodiment includes a method for personalizing and optimizing medication regime for a patient comprising:

generating a medication regime for one or more prescribed medications associated with a patient as input via a user interface;

personalizing the generated medication regime for the patient via the user interface;

generating a regime planner from the personalized medication regime for the patient via the user interface; and

forwarding a reminder to the patient to take the prescribed one or more medications from the generated regime planner via the user interface,

wherein the steps of the method are performed in accordance with a processor and a memory.

Another exemplary embodiment includes a system for personalizing and optimizing medication regime for a patient which comprises: a memory and a processor. The processor is operatively coupled to the memory and configured to implement the steps of:

generating a medication regime for one or more prescribed medications associated with a patient;

personalizing the generated medication regime for the patient;

generating a regime planner from the personalized medication regime for the patient; and

forwarding a reminder to the patient to take the prescribed one or more medications from the generated regime planner

Another exemplary embodiment includes a computer program product comprising a computer readable storage medium for storing computer readable program code which, when executed, causes a computer to:

generate a medication regime for one or more prescribed medications associated with a patient;

personalize the generated medication regime for the patient;

generate a regime planner from the personalized medication regime for the patient; and

forward a reminder to the patient to take the prescribed one or more medications from the generated regime planner.

These and other features, objects and advantages of the present invention will become apparent from the following detailed description of illustrative embodiments thereof, which is to be read in connection with the accompanying drawings.

### BRIEF DESCRIPTION OF THE DRAWINGS

FIG. 1A shows a flow diagram illustrating a method for personalizing and optimizing medication regime for a patient, according to an embodiment of the invention.

FIG. 1B shows a flow diagram exemplifying a step of the method of FIG. 1A for personalizing and optimizing medication regime for a patient, according to an embodiment of the invention.

FIG. 1C shows a flow diagram exemplifying a step of the method of FIG. 1A for personalizing and optimizing medication regime for a patient, according to an embodiment of the invention.

FIG. 1D shows a flow diagram exemplifying a step of the method of FIG. 1A for personalizing and optimizing medication regime for a patient, according to an embodiment of the invention.

FIG. 1E shows a flow diagram exemplifying a step of the method of FIG. 1A for personalizing and optimizing medication regime for a patient, according to an embodiment of the invention.

FIG. 2 depicts a computer system in accordance with which one or more components/steps of techniques of the invention which may be implemented according to an embodiment of the invention.

FIG. 3 depicts a cloud computing environment according to an embodiment of the invention.

FIG. 4 depicts abstraction model layers according to an embodiment of the present invention.

### DETAILED DESCRIPTION

Embodiments of the present invention will now be described in further detail with regard to systems and methods for allowing a user who is taking a medication to personalize and optimize their medication regime. Features of the embodiments of the present invention that can help pharmacy patients in following regimes include, for example, the number of drug ingestion sessions can be minimized, the number of pills per ingestion session can be minimized, optimization against drug half-life for interacting drugs in the prescription, personalized pill reminder messages, use of pill description and/or images in the reminders, personalized delivery channels and time for reminders, personalized medication regimes, refill, appointment and medication reminders and a personalized dispenser device. A further advantage of the features of the embodiments of the present invention including tracking if medication is misused, e.g. not taken according to protocol—not taken fast enough, not taken at all, or taken too quickly, e.g. report to authorities if opioids used too quickly. Other advantages include, for example, improves methods for pharmaceutical industry drug delivery based on context adherence preferences of populations, improves value-based care by making it scalable and adaptable for pharmacists to support their customers and bring care closer to the customer, and regime optimization and reminder services brings better health outcomes which ensures customer loyalty.

As one skilled in the art will understand, the terms “drug”, “pills” and “medication” can be used interchangeably herein.

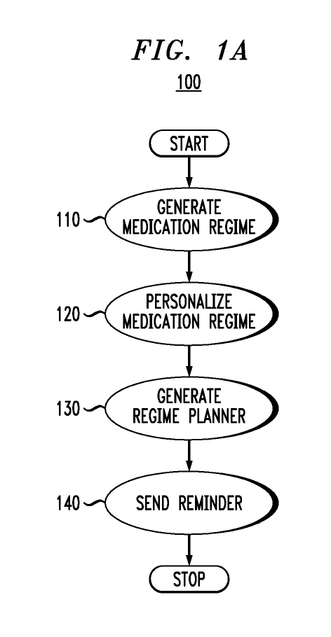

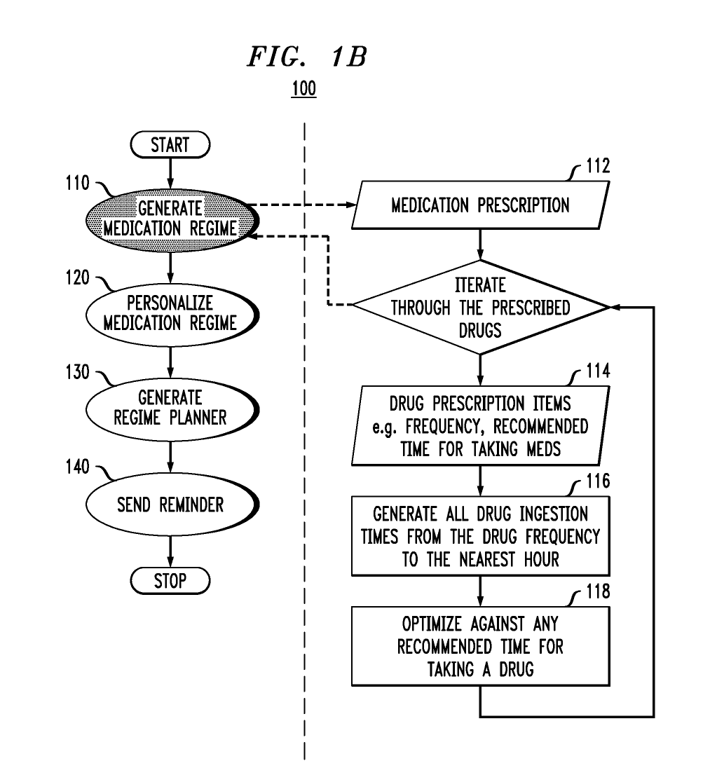

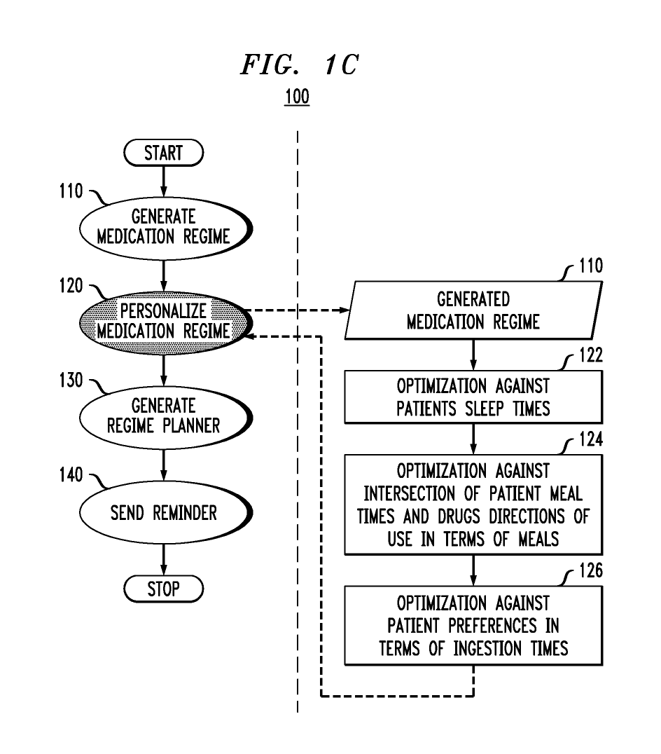

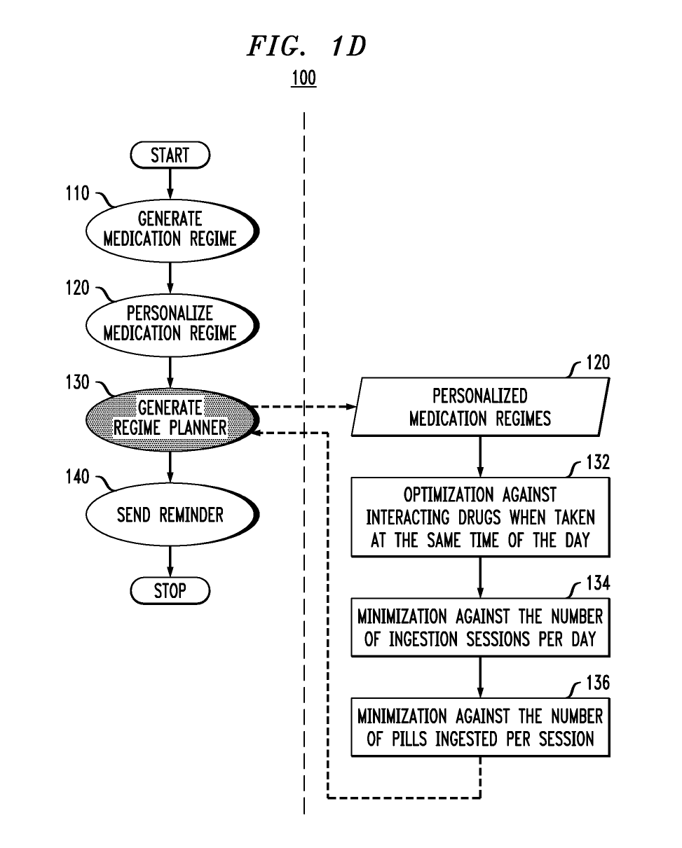

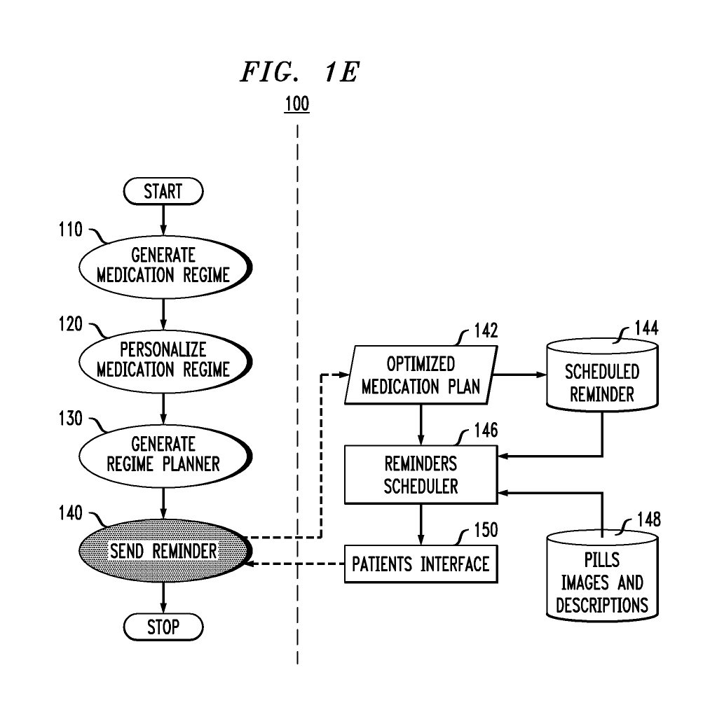

FIGS. 1A-1E show a flow diagram of a method for personalizing and optimizing medication regime for a user according to an embodiment of the present invention. With reference now to FIG. 1A, there is a flow diagram illustrating a functional overview of a personalized medication system 100 that can be implemented according to an embodiment of the present invention. First, medication regimes 110 for one or more medications to be used by a patient are generated by the personalized medication system 100. Personalization 120 of each of the generated medication regimes is inputted into the personalized medication system 100. Regime planner 130 for the personalized medication regimes is generated the personalized medication system 100. Reminders 140 are sent to the patient by the personalized medication system 100 based on the generated regime planner.

FIG. 1B is a flow diagram of an example of a medication regime 110 of personalized medication system 100 that can be implemented according to one exemplary embodiment of the present invention. Medication regime 110 involves generating all possible medication regimes as shown in the flow chart. First, all medication prescriptions for the user(s) are inputted into the personalized medication regime 100. Next, step 114 in this exemplary embodiment involves inputting all the necessary information, i.e., medication (drug) prescription items for a first medication prescription into personalized medication system 100. For example, the medication (drug) prescription items for the first medication prescription include items such as frequency of use, dosage, etc. Step 116 in this exemplary embodiment involves generating all drug ingestion times based on the medication (drug) prescription items such as the drug frequency of use to the nearest hour. Step 118 in this exemplary embodiment involves optimization of the 24 hour clock against the frequency of taking one or more prescription drugs and against any recommended time for taking a drug to come up with a range of times when the prescribed medication can be taken in personalized medication system 100. Finally, if the user is taking more than one prescribed medication, then steps 114-118 will be repeated.

FIG. 1C is a flow diagram of an example of personalization 120 based on the generated medication regime obtained from FIG. 1B of personalized medication system 100 that can be implemented according to one exemplary embodiment of the present invention. Personalization 120 in this exemplary embodiment involves personalization of the generated medication regimes 110. For example, a personalization of the generated medication regimes 110 can include, as discussed below, one or more of a patient's preferred drug ingestion time, if a patient has past regimes, whether a patient is a farmer, whether a patient goes for prayers, whether a patient sleep and meals time are known and an analysis of similar patients is done. Step 122 in this exemplary embodiment involves optimizing the generated medication regime against patient's sleep times. Step 124 in this exemplary embodiment involves optimization against intersection of patient meal times and drugs directions of use in terms of meals. Step 126 in this exemplary embodiment involves optimization against a patient's preference in terms of ingestion times of the prescribed medication.

FIG. 1D is a flow diagram of an example of a regime planner 130 for the personalized medication regimes obtained from FIG. 1C of personalized medication system 100 that can be implemented according to an exemplary embodiment of the present invention. The regime planner 130 involves optimizing the personalized medication regime 120 obtained from FIG. 1C. First, personalized medication regime 120 involves step 132 for optimizing prescribed drugs which interact if taken at the same time of the day. Personalized medication regime 120 further involves step 134 for minimization of the number of ingestion sessions per day of the prescribed medication. For example, certain prescribed medication needs to be taken during a meal or by drinking a fluid. Finally, personalized medication regime 120 further involves step 136 for minimization of the number of drugs ingested per session.

FIG. 1E is a flow diagram of an example of a reminder 140 based on the regime planner 130 obtained from FIG. 1D of personalized medication system 100 that can be implemented according to an exemplary embodiment of the present invention. The personalized reminder planner 140 involves personalization of reminder message content. For example, personalized reminder planner includes an optimized medication plan 142 which can include the following exemplary information of a patient for the reminder planner: education level of a patient, a patient's preferred message language, a patient's known age, a patient's known sex, a patient's known profession, a patient's food supply known from the weather context, and an analysis of similar patients. In one embodiment, personalized reminder planner includes an optimized medication plan 142 for scheduling reminders 144 which then sends the reminders to reminder scheduler 146. Alternatively, personalized reminder planner includes an optimized medication plan 142 which sends the reminders to the reminder scheduler 146 directly. Reminder scheduler 146 can include, for example, sending a reminder through patient's interface 150 by way of, for example, a text message to a patient thirty minutes prior to the pill ingestion time. In addition, reminder scheduler 146 can include in the reminder message one or more of a drug image, drug description and direction of use obtained from pill images and descriptions 148. Other examples of reminder scheduler 146 can include, for example, sending a reminder through patients interface 150 by way of, for example, an e-mail to the patient, use of light flickering for a hearing impaired patient, using a sound alarm for a visually impaired patient, posting on the patient's facebook page, use of the whatsapp of a patient, and the like.

In another embodiment of the present invention, a smart medicine container can be used in connection with the regime planner 130 and reminders 140 to dispense only the desired number of pills to a patient. In one exemplary embodiment, the personalized medication regime 100 can be adapted to control the interlocking mechanism of the smart medicine container, e.g., enabling interlocks and/or notification for bottles at times and for medication as determined optimal by the personalized medication regime 100. For example, the personalized medication regime 100 can be adapted to control the number of pills and the time of dispensing the pills to the patient from the smart medicine container.

For example, a smart medicine container can have a dispenser having means to dispense a desired number of pills from a bulk supply of pills contained in the dispenser. The dispenser comprises of storage compartment having bulk supply of pills and having a discharge port emptying into counting compartment. The counting compartment contains first and second conveyors moving at first and second speed; wherein the second speed is greater than the first speed thereby enabling pill separation; the second conveyor discharges pills into dispensing compartment. Sensors are strategically placed along the conveyors to count pills discharged into dispensing compartment. A pill recovery system and apparatus is disposed inside the dispenser having means to recover pills remaining on conveyors upon completion of a dispensation cycle and deposit recovered pills back into the storage compartment for use in future dispensation cycles. A docking station having receptacles to accommodate dispenser is provided. The docking station has communication ports enabling two-way communication with a personal computer. The dispenser has multiple security features including locking mechanisms at inlet and outlet; and internal circuitry that is responsive to ‘disable’ electronic the signal originating from dispenser's internal clock and remote server in communication link with the dispenser.

In another embodiment of the present invention, a home pill dispensing system can be used in connection with the regime planner 130 and reminders 140 to dispense only the desired number of pills to a patient. In one exemplary embodiment, a home pill dispensing system can provide over dosage protection with the ability to provide medication doses within selected periods of time. The home pill dispensing system can be adapted to have the optimized medication regime plan controlling the dispensing mechanism of the system in terms of the number of pills and the time of dispensing.

For example, a home pill dispensing system can comprise a housing unit defining a first and second opening, a medication containment unit, a request signal generating unit, a dispensing unit, a medication collection unit, and a control unit. The medication containment unit includes a plurality of sealed dosage compartments received within the first opening in the housing. Each of the dosage compartments has an associated time period during which the dosages may be dispensed. The request signal generating unit generates a request signal. The dispensing unit causes medication doses that are requested within their respective time periods to be dispensed from the device through the second opening. The medication collection unit prevents medication doses that are not requested within their respective time periods from being dispensed from the device. The control unit controls the dispensing unit and the medication collection unit, responsive to the request signal and the time periods.

In another embodiment of the present invention, an automatic inventory and replenishment system can be used in connection with the regime planner 130 and reminders 140. For example, regime planner 130 can have directions of use of the prescribed medication which can be used to inform stocking of the patient's refrigerator. In addition, some prescribed medications may require a unique diet and the automatic inventory and replenishment system can be used to ensure their availability. Also, the automatic inventory can be adapted to avoid misuse of prescribed medication, e.g., not taking the prescribed medication according to protocol, not taking the prescribed medication fast enough, not taking the prescribed medication at all, or too quickly, e.g. report to authorities if opioids are used too quickly.

For example, a household consumable item automatic replenishment system will automatically maintain a desired inventory of household consumable items. The household consumable item automatic replenishment system can have a refrigerator compartment having an indoor access and an outdoor access and an unrefrigerated compartment having an indoor access and an outdoor access. An automatic inventory system has a plurality of sensors configured to provide information representative of an inventory of the refrigerated compartment and the unrefrigerated compartment. An inventory processor is coupled to the sensors to process the information representative of the inventory of the refrigerated compartment and the unrefrigerated compartment, so as to make a list of items which are to be replenished. An automatic ordering system comprises a telecommunications device coupled to cooperate with the inventory processor to communicate at least a portion of the list to at least one vendor.

In another embodiment of the present invention, a system for managing expiration-dated products utilizing an electronic receipt can be used in connection with the regime planner 130 and reminders 140. For example, the system can be adapted for a household setting refrigerator to alert the user on use of out of date medication, compromised medication (e.g. not refrigerated properly), etc.

For example, a system by which information concerning the shelf-life limitations of a particular product item is made available to the purchaser electronically; that the electronically recorded shelf-life limitation information is provided to, or made accessible by, the purchaser; that the electronically recorded shelf-life limitation information is communicated to a computer equipped with a microprocessor, or to a computer system network, accessible by the purchaser, programmed to receive the shelf-life limitation data for each product for that purchaser; that the computer or purchaser-accessible computer system network, is further programmed to provide the purchaser with on-screen and/or printed reports of various formats that list the items purchased and the corresponding shelf-life limitation information; and that the computer or purchaser-accessible computer system network provides interactivity with the purchaser to allow the purchaser to identify further information to the computer/network, such as identifying location information of each particular product item, and the inventory status of each particular product item, e.g., whether the product has been opened or has been discarded.

In another embodiment, the personalized medication regime can also be expanded for optimization of commercial setting with multiple patients, e.g., a hospital station for drug dispensing.

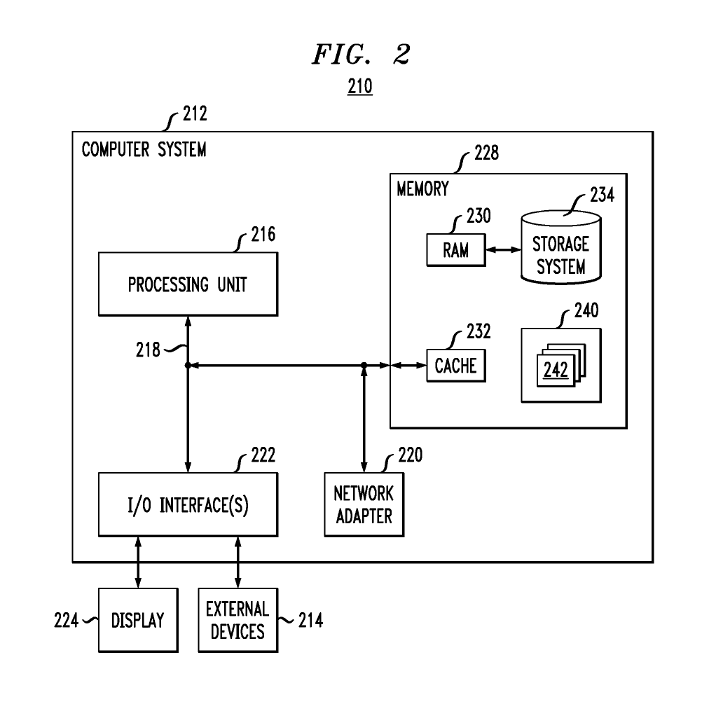

One or more embodiments can make use of software running on a computer or workstation. With reference to FIG. 2, in a computing node 210 there is a system/server 212, which is operational with numerous other general purpose or special purpose computing system environments or configurations. Examples of well-known computing systems, environments, and/or configurations that may be suitable for use with system/server 212 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

System/server 212 may be described in the general context of computer system executable instructions, such as program modules, being executed by a computer system. Generally, program modules may include routines, programs, objects, components, logic, data structures, and so on that perform particular tasks or implement particular abstract data types. System/server 212 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 2, system/server 212 is shown in the form of a computing device. The components of system/server 212 may include, but are not limited to, one or more processors or processing units 216, system memory 228, and bus 218 that couples various system components including system memory 228 to processor 216.

Bus 518 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

System/server 212 typically includes a variety of computer system readable media. Such media may be any available media that is accessible by system/server 212, and it includes both volatile and non-volatile media, removable and non-removable media.

The system memory 228 can include computer system readable media in the form of volatile memory, such as random access memory (RAM) 230 and/or cache memory 232. System/server 212 may further include other removable/non-removable, volatile/nonvolatile computer system storage media. By way of example only, storage system 234 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 218 by one or more data media interfaces.

As depicted and described herein, memory 228 may include at least one program product having a set (e.g., at least one) of program modules that are configured to carry out the functions of embodiments of the invention. A program/utility 240, having a set (at least one) of program modules 242, may be stored in memory 228 by way of example, and not limitation, as well as an operating system, one or more application programs, other program modules, and program data. Each of the operating system, one or more application programs, other program modules, and program data or some combination thereof, may include an implementation of a networking environment. Program modules 242 generally carry out the functions and/or methodologies of embodiments of the invention as described herein.

System/server 212 may also communicate with one or more external devices 214 such as a keyboard, a pointing device, an external data storage device (e.g., a USB drive), display 224, one or more devices that enable a user to interact with system/server 212, and/or any devices (e.g., network card, modem, etc.) that enable system/server 212 to communicate with one or more other computing devices. Such communication can occur via I/O interfaces 222. Still yet, system/server 212 can communicate with one or more networks such as a LAN, a general WAN, and/or a public network (e.g., the Internet) via network adapter 220. As depicted, network adapter 220 communicates with the other components of system/server 212 via bus 218. It should be understood that although not shown, other hardware and/or software components could be used in conjunction with system/server 212. Examples include, but are not limited to, microcode, device drivers, redundant processing units, external disk drive arrays, RAID systems, tape drives, and data archival storage systems, etc.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

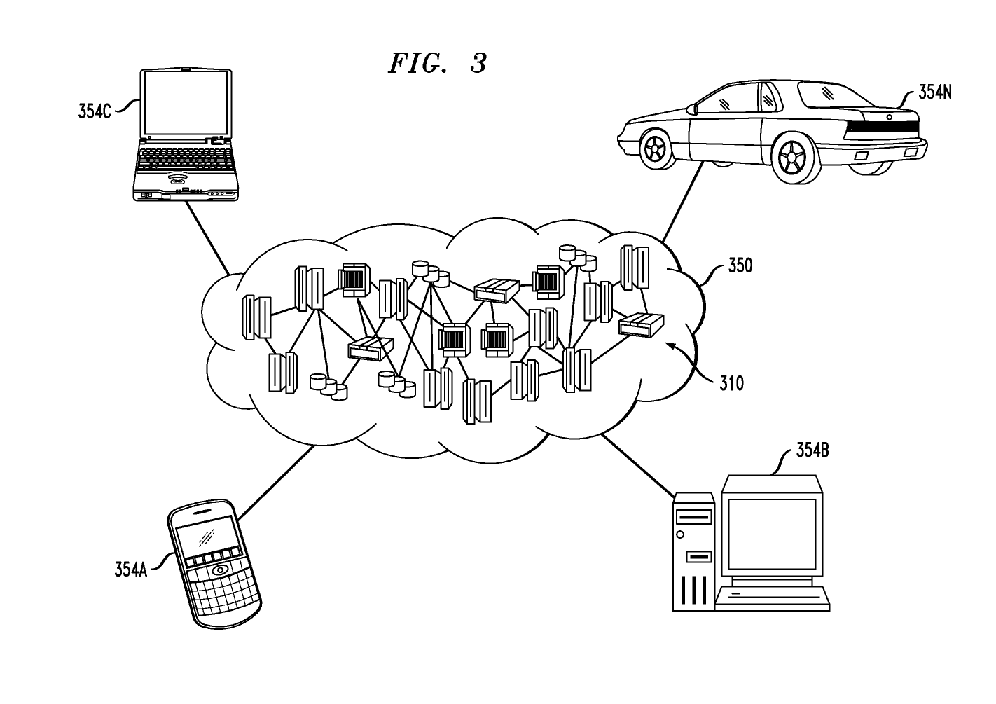

Referring now to FIG. 3, illustrative cloud computing environment 350 is depicted. As shown, cloud computing environment 350 includes one or more cloud computing nodes 310 with which local computing devices used by cloud consumers, such as, for example, personal digital assistant (PDA) or cellular telephone 354A, desktop computer 354B, laptop computer 354C, and/or automobile computer system 354N may communicate. Nodes 310 may communicate with one another. They may be grouped (not shown) physically or virtually, in one or more networks, such as Private, Community, Public, or Hybrid clouds as described hereinabove, or a combination thereof. This allows cloud computing environment 350 to offer infrastructure, platforms and/or software as services for which a cloud consumer does not need to maintain resources on a local computing device. It is understood that the types of computing devices 354A-N shown in FIG. 3 are intended to be illustrative only and that computing nodes 310 and cloud computing environment 350 can communicate with any type of computerized device over any type of network and/or network addressable connection (e.g., using a web browser).

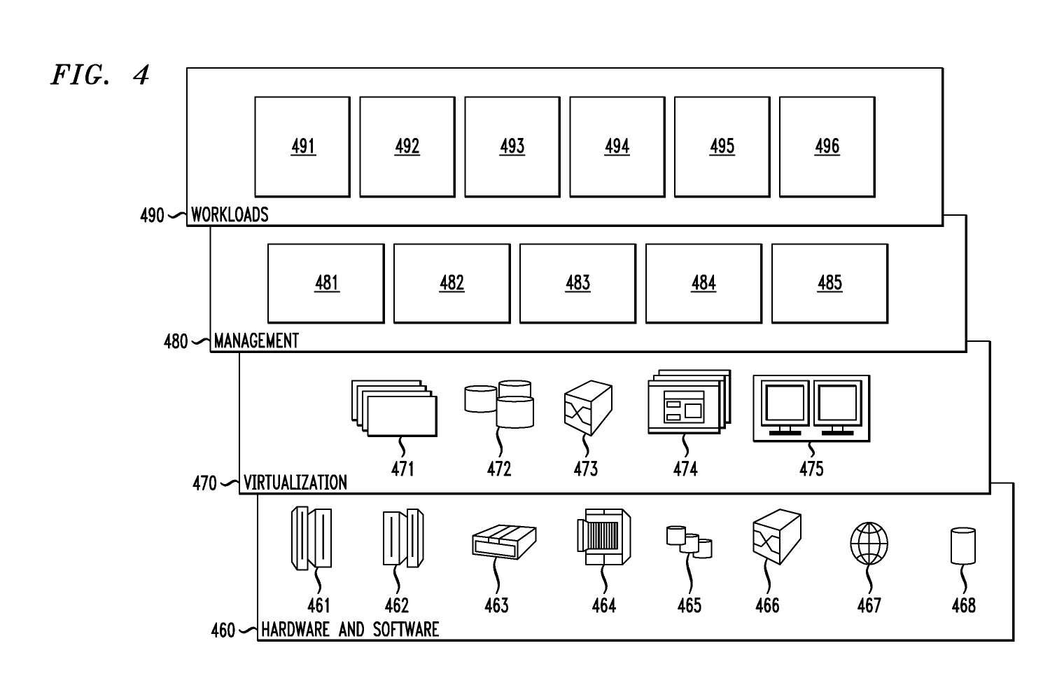

Referring now to FIG. 4, a set of functional abstraction layers provided by cloud computing environment 350 (FIG. 3) is shown. It should be understood in advance that the components, layers, and functions shown in FIG. 4 are intended to be illustrative only and embodiments of the invention are not limited thereto. As depicted, the following layers and corresponding functions are provided:

Hardware and software layer 460 includes hardware and software components. Examples of hardware components include: mainframes 461; RISC (Reduced Instruction Set Computer) architecture based servers 462; servers 463; blade servers 464; storage devices 465; and networks and networking components 466. In some embodiments, software components include network application server software 467 and database software 468.

Virtualization layer 470 provides an abstraction layer from which the following examples of virtual entities may be provided: virtual servers 471; virtual storage 472; virtual networks 473, including virtual private networks; virtual applications and operating systems 474; and virtual clients 475.

In one example, management layer 480 may provide the functions described below. Resource provisioning 481 provides dynamic procurement of computing resources and other resources that are utilized to perform tasks within the cloud computing environment. Metering and Pricing 482 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 483 provides access to the cloud computing environment for consumers and system administrators. Service level management 484 provides cloud computing resource allocation and management such that required service levels are met. Service Level Agreement (SLA) planning and fulfillment 485 provide pre-arrangement for, and procurement of, cloud computing resources for which a future requirement is anticipated in accordance with an SLA.

Workloads layer 490 provides examples of functionality for which the cloud computing environment may be utilized. Examples of workloads and functions which may be provided from this layer include: transaction data capture 491; blockchain computation 492; data analytics processing 493; risk assessment 494; alert processing 495; and ameliorative/corrective/remedial action implementation 496, which may perform various functions described above.

Embodiments of the present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Embodiments of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The flowchart diagrams in the figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the diagrams, and combinations of blocks in the diagrams, can be implemented by special purpose hardware-based systems that perform the specified functions or acts or carry out combinations of special purpose hardware and computer instructions.

Although illustrative embodiments have been described herein with reference to the accompanying drawings, it is to be understood that the invention is not limited to those precise embodiments, and that various other changes and modifications may be made by one skilled in art without departing from the scope or spirit of the invention.

What is claimed is:

1. A method for personalizing and optimizing a medication regime for a patient comprising:
generating a medication regime for one or more prescribed medications associated with a patient as input via a user interface;
personalizing the generated medication regime for the patient via the user interface;
generating a regime planner from the personalized medication regime for the patient via the user interface; and
forwarding a reminder to the patient to take the prescribed one or more medications from the generated regime planner via the user interface,
wherein the steps of the method are performed in accordance with a processor and a memory.

2. The method of claim 1, wherein the step of generating a medication regime comprises inputting via the user interface a frequency of use by the patient of the prescribed medication, and recommended times for taking the prescribed medication by the patient.

3. The method of claim 2, further comprising generating all drug ingestion times for the patient based on the frequency of use of the prescribed medication to the nearest hour.

4. The method of claim 3, further comprising optimizing in a twenty four time period against the frequency of taking the prescribed medication and against any recommended time for taking a drug to arrive at a range of times when the prescribed medication can be taken by the patient.

5. The method of claim 1, wherein the step of personalizing comprises optimizing the generated medication regime against a patient's sleep times.

6. The method of claim 1, wherein the step of personalizing comprises optimizing against intersection of a patient's meal times and drugs directions of use in terms of meals.

7. The method of claim 1, wherein the step of personalizing comprises optimizing against a patient's desired ingestion time of the prescribed medication.

8. The method of claim 1, wherein the step of generating a medication regime comprises generating a medication regime for more than one prescribed medications associated with the patient, and the step of generating the regime planner comprises optimizing the prescribed medication which interact if taken at the same time of the day.

9. The method of claim 1, wherein the step of generating the regime planner comprises minimizing a number of ingestion sessions per day of the prescribed medication.

10. The method of claim 1, wherein the step of generating a medication regime comprises generating a medication regime for more than one prescribed medications associated with the patient, and the step of generating the regime planner comprises minimizing a number of the more than one prescribed medications ingested per session.

11. The method of claim 1, wherein the step of forwarding the reminder to the patient comprises one of sending a text message to the patient, sending an e-mail to the patient, posting the reminder on facebook, sounding an alarm for a hearing impaired patient, and flashing a light for a visually impaired patient.

12. The method of claim 1, wherein the step of forwarding the reminder to the patient further comprises including one or more of an image of the prescribed medication and a description of the prescribed medication.

13. A system comprising:
a memory and a processor operatively coupled to the memory and configured to implement the steps of:
generating a medication regime for one or more prescribed medications associated with a patient;
personalizing the generated medication regime for the patient;
generating a regime planner from the personalized medication regime for the patient; and
forwarding a reminder to the patient to take the prescribed one or more medications from the generated regime planner.

14. The system of claim 13, wherein generating the medication regime comprises inputting a frequency of use by the patient of the prescribed medication, and recommended times for taking the prescribed medication by the patient.

15. The system of claim 13, wherein personalizing comprises one or more of:
optimizing the generated medication regime against a patient's sleep times;
optimizing against intersection of a patient's meal times and drugs directions of use in terms of meals; and
optimizing against a patient's desired ingestion time of the prescribed medication.

16. The system of claim 13, wherein generating the medication regime comprises one or more of:
generating a medication regime for more than one prescribed medications associated with the patient, and generating the regime planner comprises optimizing the prescribed medication which interact if taken at the same time of the day;
minimizing a number of ingestion sessions per day of the prescribed medication; and
generating a medication regime for more than one prescribed medications associated with the patient, and the regime planner comprises minimizing a number of the more than one prescribed medications ingested per session.

17. The system of claim 13, wherein the reminder to the patient comprises one of sending a text message to the patient, sending an e-mail to the patient, posting the reminder on facebook, sounding an alarm for a hearing impaired patient, and flashing a light for a visually impaired patient.

18. The system of claim 13, wherein the reminder to the patient further comprises including one or more of an image of the prescribed medication and a description of the prescribed medication.

19. The system of claim 13, which is further coupled to a medication dispensing container.

20. A computer program product comprising a computer readable storage medium for storing computer readable program code which, when executed, causes a computer to:
generate a medication regime for one or more prescribed medications associated with a patient;
personalize the generated medication regime for the patient;
generate a regime planner from the personalized medication regime for the patient; and
forward a reminder to the patient to take the prescribed one or more medications from the generated regime planner.
